(19) United States
(12) Patent Application Publication
(10) Pub. No.: US 2025/0281195 A1
(43) Pub. Date: Sep. 11, 2025

(54) PROTECTIVE MOUTH ASSEMBLY AND METHODS

(71) Applicant: Nationwide Children's Hospital, Columbus, OH (US)

(72) Inventors: Kris R. Jatana, Columbus, OH (US); Charles A. Elmaraghy, Columbus, OH (US)

(21) Appl. No.: 19/071,492

(22) Filed: Mar. 5, 2025

Related U.S. Application Data
(63) Continuation of application No. 29/931,163, filed on Mar. 5, 2024.
(60) Provisional application No. 63/561,515, filed on Mar. 5, 2024.

(51) Int. Cl. A61B 17/24; A61B 17/00; A61B 17/02

(52) U.S. Cl. CPC A61B 17/24; A61B 17/0218; A61B 2017/0073

(57) ABSTRACT

A protective mouth assembly for covering a patient's lips and adjacent skin or intraoral mucosa to help reduce or prevent tissue injury during surgical procedures, the protective mouth assembly includes an inner portion, an outer portion, and a base portion such that the base portion spaces the inner portion from the outer portion to form an open cavity or channel therebetween.

### CROSS REFERENCES TO RELATED APPLICATIONS

The following application claims priority under 35 U.S.C. § 119(e) to U.S. Provisional Patent Application Ser. No. 63/561,515 filed Mar. 5, 2024, entitled PROTECTIVE MOUTH ASSEMBLY AND METHODS and is a continuation application under 35 U.S.C. § 120 to U.S. Design patent application Ser. No. 29/931,163 that was filed on Sep. 3, 2024. The above-identified applications are incorporated herein by reference in their entireties for all purposes.

### TECHNICAL FIELD

The present disclosure generally relates to a protective mouth assembly and method of use, and more particularly to a protective mouth assembly for covering a patient's lips and skin to reduce or prevent tissue injury during surgical procedures.

### BACKGROUND

During many transoral surgeries, a need exists to introduce surgical instruments into the oropharyx, larynx, hypopharynx, nasopharynx, or oral cavity of a patient. During insertion, a patient's mouth is typically held open using a bite block, mouth gag, or other type of retractor. An example of an oral retractor, how it is used, and how it is constructed is further described and shown in U.S. Pat. No. 4,024,859 entitled MOUTH GAG HAVING THREE-DIMENSIONAL ALVEOLUS RETRACTOR ADJUSTABILITY and U.S. Pat. No. 3,241,550 entitled MOUTH RETRACTOR. To help reduce or prevent mechanical, thermal, or electrical injuries to the lips and adjacent skin or intraoral mucosa, protective mouth assemblies has been invented to provide such protection by in the Applicants' earlier U.S. Pat. No. 11,284,968 entitled PROTECTIVE MOUTH ASSEMBLY AND METHOD OF USE. All the aforementioned patents in this paragraph are incorporated herein by reference in their entireties.

Surgical instruments, such as sharp or heated instruments, for example, scalpels and electrocautery, respectively, are generally used in performing oral or throat surgery, such as a tonsillectomy and/or adenoidectomy. In some oral surgeries, drills are used, which can contain a revolving shaft. Other types of surgical instruments are known to have a potential injury risk. Once the patient's mouth is secured open using a retractor, the patient's lips, adjacent tissue, and/or skin become exposed to such instruments, and at times unfortunately results inoral commissure/lip trauma from mechanical injury, lacerations, and/or burns during many common procedures. At times, resulting tissue injuries include, but are not limited to oral commissure/lip trauma from mechanical injury, lacerations, and/or burns, bruising, redness, and scars can lead to permanent cosmetic deformities during surgical procedures involving the mouth or throat.

### SUMMARY

One aspect of the present disclosure includes a protective mouth assembly for covering a patient's lips and adjacent skin or intraoral mucosa to help reduce or prevent tissue injury during surgical procedures, the protective mouth assembly includes a first member spaced from a second member. The first and second members are coupled and spaced by a connecting member. The connecting member has a first end and a second end spaced by an undulating surface. Projecting from the first and second ends is a biasing arm for integrally coupling the first member with the undulating surface and the second member to the undulating surface.

Another aspect of the present disclosure includes a protective mouth assembly comprising a first member spaced from a second member, the first and second members are coupled and spaced by a connecting member. The connecting member has a first end and a second end spaced by a connecting portion surface. Projecting from the first and second ends is a biasing arm having a plurality of curvatures, the biasing arm for integrally coupling the first member with the connecting portion surface and the second member to the connecting portion surface.

While yet another aspect of the present disclosure includes a protective mouth assembly comprising a polymeric member for straddling a lip surface of a patient during use, the polymeric member having an outer portion arrangement coupled to and spaced from an inner portion arrangement by a base portion to form a channeled cavity therein. The channeled cavity further comprises a converging diverging cavity that is formed by the inner portion arrangement and the outer portion arrangement. A slot formed within and through the surface of the inner portion arrangement forms two flexible and independently movable flaps.

Another aspect of the present disclosure includes a protective mouth assembly having a polymeric member for straddling a lip surface of a patient during use. The polymeric member comprises an outer portion arrangement coupled to and spaced from an inner portion arrangement by a base portion to form a cavity therein. A slot forms within the inner portion arrangement forming two flexible and independently movable flaps.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals, unless otherwise described refer to like parts throughout the drawings and in which:

FIG. 1A is an image of a protective mouth assembly positioned in an oral cavity of a patient in accordance with one example embodiment of the present disclosure;

FIG. 1B is an image of a protective mouth assembly positioned in an oral cavity of a patent in accordance with another example embodiment of the present disclosure;

FIG. 1C is an upper front right elevation view of a protective mouth assembly in accordance with another example embodiment;

FIG. 2 is an upper front left elevation view of a protective mouth assembly in accordance with one example embodiment;

FIG. 3 is a lower rear left elevation view of the protective mouth assembly in accordance with one example embodiment;

FIG. 4 is a lower rear right elevation view of the protective mouth assembly in accordance with one example embodiment;

FIG. 5 is a bottom plan view of the protective mouth assembly in accordance with one example embodiment;

FIG. 6 is a top plan view of the protective mouth assembly in accordance with one example embodiment;

FIG. 7 is a right-side view of the protective mouth assembly in accordance with one example embodiment;

FIG. 8 is a left-side view of the protective mouth assembly in accordance with one example embodiment;

FIG. 9 is a top perspective view of the protective mouth assembly in accordance with one example embodiment;

FIG. 10 is a bottom perspective view of the protective mouth assembly in accordance with one example embodiment;

FIG. 11 is a magnified section view of FIG. 6 along section lines 11-11; and

FIG. 12 is a protective mouth assembly, illustrating that protective mouth assembly is dimensioned and configured to react by increasing or decreasing the channel between the inner and/or outer portions based on the position and force applied by a medical instrument.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure generally relates to a protective mouth assembly and method of use, and more particularly to a protective mouth assembly for covering a patient's lips and adjacent skin or intraoral mucosa to help reduce or prevent tissue injury during surgical procedures.

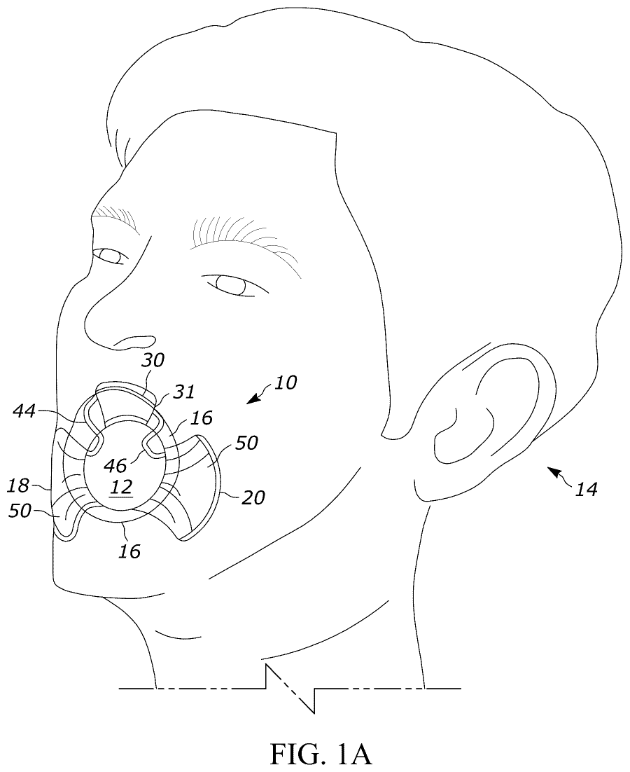

FIG. 1A illustrates a protective mouth assembly 10 (see FIG. 1C) positioned in a patient's oral cavity 12 in accordance with one example embodiment of the present disclosure. FIG. 1B illustrates a protective mouth assembly 10 (see FIG. 1C) positioned in a patient's oral cavity 12 in accordance with another example embodiment of the present disclosure. It should be appreciated by one of ordinary skill in the art that the positioning of the mouth assembly 10 can be rotated within the patient's oral cavity. In yet another example embodiment, the protective mouth assembly 10 is rotated 180 degrees when positioned in the patient from what is illustrated in FIGS. 1A and 1B.

The protective mouth assembly 10 in the illustrated example embodiment is designed to protect a patient's lips and further can be customized such that the single assembly will fit any size mouth opening of the oral cavity of any size patient. Moreover, the assembly 10 can be used with or without mouth retractors and the assembly is placed at the beginning of any surgical procedure in this region of the body. Such procedures include, but are not limited to, tonsillectomies, adenoidectomy, dental procedures, cleft lip or cleft palate procedures, and the like.

In accordance with another example embodiment, the assembly 10 is constructed in such that it is disposable after a single use. That is, the construction takes into consideration reducing fabrication costs associated with manufacturing each assembly 10. Thus, the opportunity to spread bacteria, germs or diseases is eliminated, since each assembly is disposable and used only once by one patient before being discarded. Moreover, such construction eliminates the need for sterilization. An example of such material used to form the assembly 10 includes a polymeric material. In another example embodiment, the polymeric material has a high enough durometer to resist melting when in contact with a medical instrument, but a low enough durometer to allow some degree of elasticity to be compressed and expand to be secured to a patient's oral cavity. In one example embodiment, the polymeric material is formed in a mold having a durometer ranging between 40 and 90 on a shore A scale. In another example embodiment, the polymeric material is polyethylene, polypropylene, a natural rubber material, silicone, and/or any combination thereof.

In an alternative example embodiment, the assembly 10 is constructed from a material conducive to sterilization such as medical grade silicone. That is, the assembly 10 will withstand heat, pressure, washing and other operations used in sterilizing medical devices.

FIG. 1A illustrates the assembly 10 positioned in the oral cavity 12 of a patient 14 in accordance with one example embodiment. The assembly 10 protects a portion of patient's lip surface 15, commissure, and skin from abrasions, mechanical trauma, and/or lacerations when various instruments are inserted during surgical procedures. In addition, the assembly 10 is constructed from a substantially thermal transfer-resistant material in order to act as a thermal barrier (such as electrocautery burns in the range of 400° F./205° C.) between the surgical instruments and the skin. The assembly 10 is used with or without a mouth retractor. The example embodiment of FIG. 1A is used and inserted into the opening of the oral cavity 12 with or without the use of a retractor.

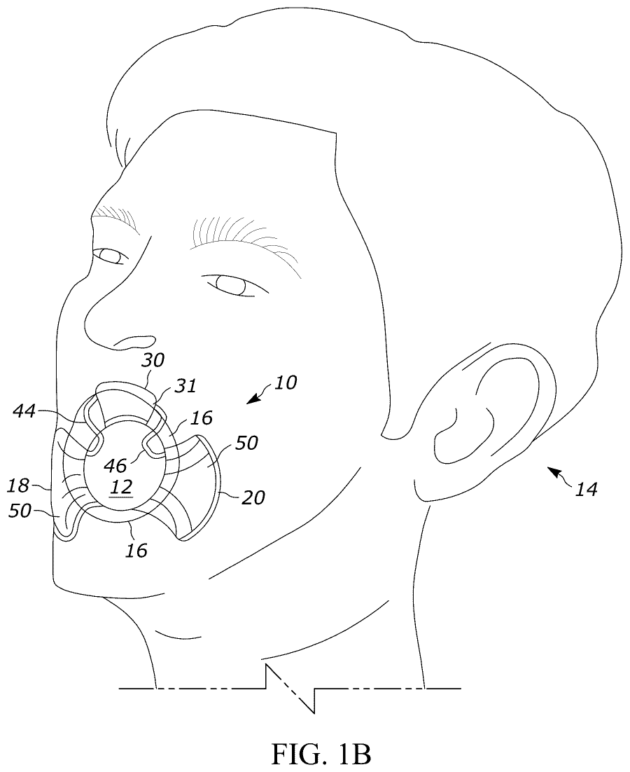

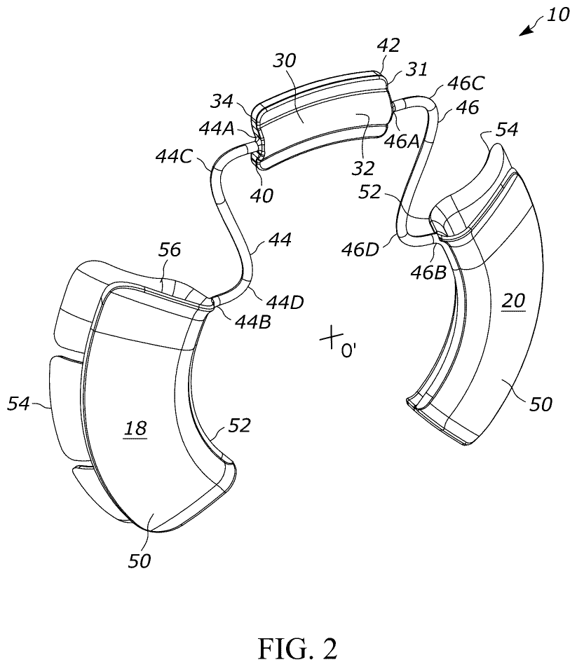

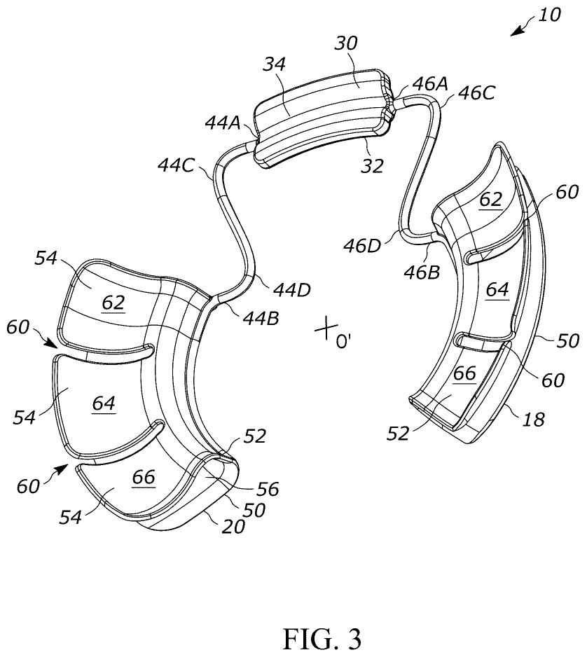

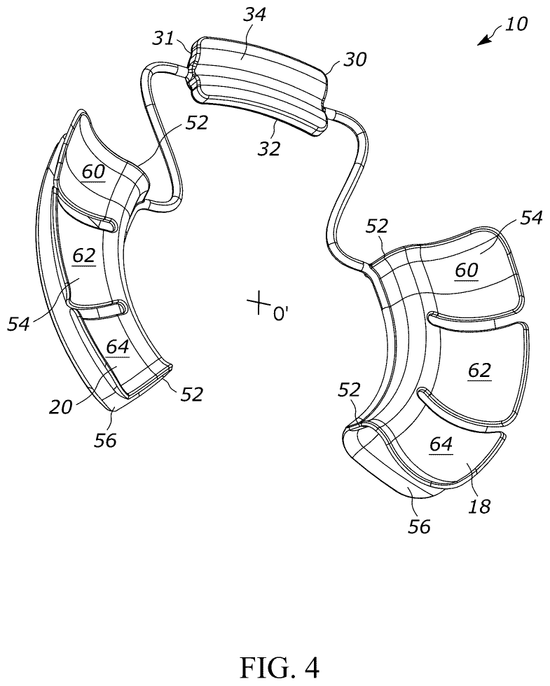

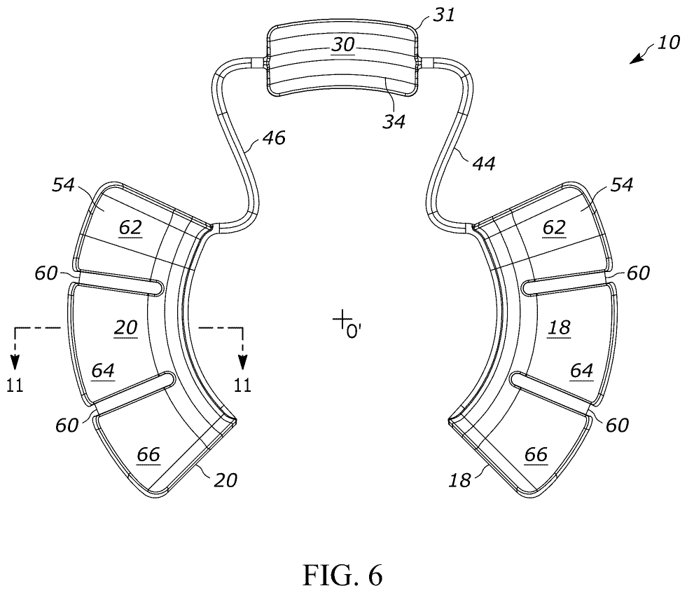

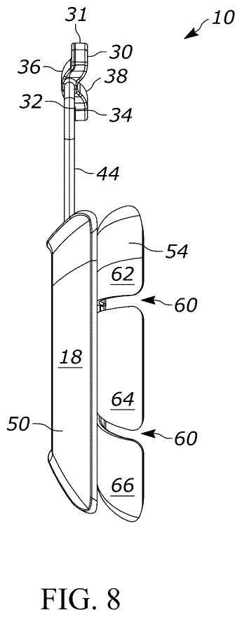

FIGS. 1B through 10 further illustrate the assembly 10 in various views in accordance with the one example embodiment. The assembly 10 in one example embodiment is a single part molded thermal transfer-resistant plastic radially biased to a substantially ovular or obround shape (see the dash-dot line of FIG. 5) for opening a mouth as illustrated in FIG. 1B. Advantageously, the construction and design of the assembly 10 allows its shape to be biased into a plurality of positions. The ovular shape of the assembly 10 can be biased under a force by the medical professional inward in the direction of arrows A to reduce the size of the obround/ovalular shape and form a first position as it is inserted into the patient's oral cavity. Upon release of the assembly 10 by the medical professional, the assembly springs outwardly in the direction of arrows B to a second position and locks within the oral cavity 12 of the patient. It should be appreciated by one of ordinary skill in the art after reviewing the present disclosure and drawings that a plurality of positions could be formed by the assembly 10 without departing from the spirit and scope of the present disclosure.

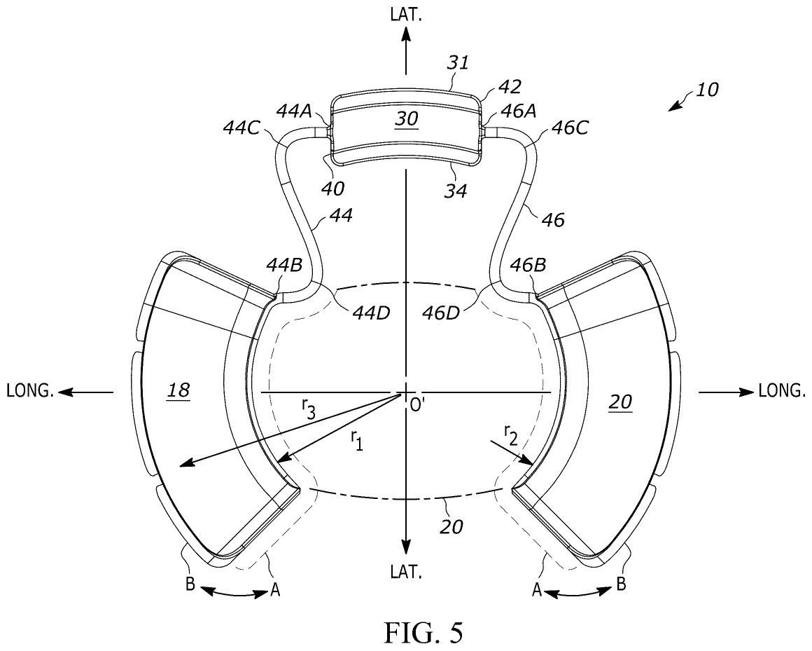

To obtain the above second, locking position of the assembly 10 into a patient's oral cavity 12, the assembly 10 is designed to include a first member 18 coupled to and spaced to a second member 20 by a connecting member 30 in one example embodiment of the present disclosure. In this example embodiment, the connecting member 30 includes an undulating surface 31 having a first side and a second side, respectively 32, 34. The undulating surface 31 of connecting member 30 further includes a constant thickness “t” forming a mound 36 which acts as a biasing member on the first side 32 and contiguous trough 38 on the second side 34 as shown in FIG. 7. In another example embodiment, the connecting member 30 comprises a substantially planar surface that covers at least part of a patient's upper lip when the assembly 10 is in use. The mound 36 in one example embodiment acts as a pillow or cushion to protect the patient's skin or upper lip from contact with instruments and/or air lines that are used during procedures being performed by medical professionals.

The connecting member 30 further comprises first and second ends 40, 42 respectively projecting respective biasing arms 44 and 46 The biasing arms 44, 46 couple the undulating surface 31 to their respective first and second members 18 and 20. In the illustrated example embodiment, the biasing arms 44, 46 have a uniform radial surface that is formed with the undulating surface 31 at first ends 44A, 46A and to the connecting member 30 at second ends 44B, 46B to their respective connecting member 18, 20. Near the first ends 44A, 46A, the biasing arms 44, 46 include a minor curvature 44C, 46C that are planar about a lateral axis LAT. and longitudinal axis LONG shown in FIG. 5 having an intersection at the arcuate foci O′ of r₁ and r₂ formed by the first and second members 18, 20 shown in FIG. 5. Stated another way, the biasing arms 44, 46 remain in a single plane without movement in situ in the central axis Cₐₓᵢₛ illustrated in FIG. 7.

However, it would be appreciated by one of ordinary skill in the art after reviewing this disclosure and accompanying drawings that during insertion, the biasing arms 44, 46 are flexed out of the plane formed by the LAT and LONG axes, advantageously allowing the first member 18, second member 20 and undulating surface 31 of the connecting member 30 to be in the same or multiple independent planes when inserted into the patient's cavity 12 because the biasing arms act as a connecting weakness (through the design of a smaller cross-sectional area) than and to the members and undulating surface. Moreover, each of the first member 18, second member 20, and undulating surface 31 can function independently without pulling on the other when tension is applied to either of the other 2 portions that protect the right and left lip commissure of the lip surface 16 when installed in the cavity 12. Advantageously, the “S” shape of the biasing arms 44, 46 allow for stabilization of the assembly 10 such that the internal portions of the assembly 10 remain stationary while the outer portion arrangement 50 moves. The shape and configuration of the biasing arms 44, 46 also allow for first and second members 18, 20 to fold away from the lips or mouth of the patient where the assembly 10 is inserted when medial to lateral directed force toward the lip commissure is applied to the assembly 10, thus reducing the risk of injury to the commissure within the mouth of a patient. The S-shape twist generated in the biasing arms 44, 46 further helps secure the assembly in the patient's cavity against movement when contacted by instruments, clamps, and/or hoses during procedures performed by medical professionals.

The minor curvature 44C, 46C and major curvature 44D, 46D, provide convenient flexing of the first and second members 18, 20 for inserting in the cavity 12, such that a medical professional is able to more easily bias the arms 44, 46 together. This convenience is achieved by positioning a thumb and index finger into major curvature 44D, 46D and pinching the arms 44, 46 together such that the ovular shape of first 18 and second 20 members is reduced, whilst the assembly 10 is inserted into the cavity (see dashed lines of FIG. 5) then released to stabilize on the patient's lip surface 16. The minor curvatures 44C, 46C, advantageously keep the biasing arms 44, 46 away and out of contact of the lip surface 16.

In one example embodiment, the major curvatures 44D, 46D have a larger radius than the minor curvatures 44C, 46C. In another example embodiment, the minor curvatures 44C, 46C have the same radius of the major curvature 44D, 46D. The ‘S’ shaped concept on the biasing arms 44 and 46 between the undulating surface 31 and first and second members 18, 20 is designed in a way that when the three pieces 18, 31, and 20 are moved into differing planes from each other, it minimizes the amount of the arms 44 and 46 from protecting outwardly of the cavity 12. This favorably reduces any potential obstruction for the surgeon.

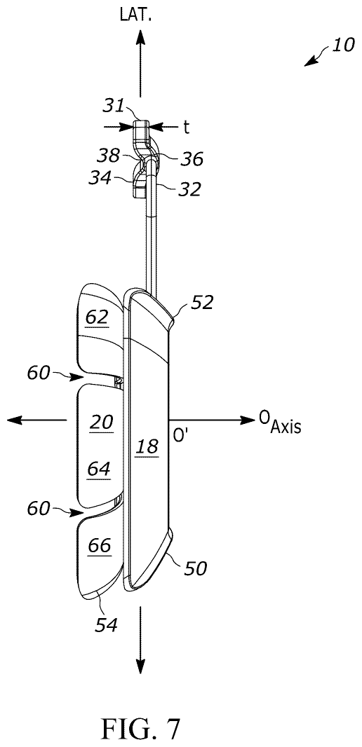

The first and second members 18, 20 respectively are mirror images of each other about the lateral axis LAT. In one example embodiment, the first and second members 18, 20 comprise a u-shaped or parabolic cross-section about the lateral axis LAT and longitudinal axis LONG., formed by an outer portion arrangement 50 and a spaced inner portion arrangement 54, the inner and outer portion arrangements uniformly connected by a base portion 52 forming a trough cavity 56 therein (see FIGS. 9-11). The outer portion arrangement 50 during use is positioned over the lip surface 16. The inner portion arrangement 54 during use is positioned on the inside of the lip surface 16, while the base portion 52 straddles the lip surface, contacting the patient's lip in various pinch or holding points, “a”, “b”, and “c” formed by the cavity 56.

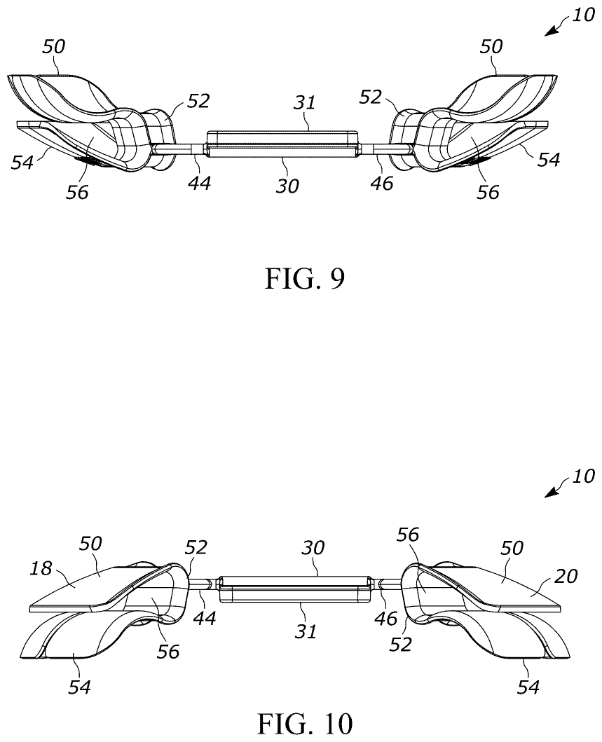

The mold of the assembly in one example embodiment includes a variable cavity 56 that includes pinch point “a” and pinch point “c” that have a greater space between the inner and outer members than the space between the same members at pinch point “b”. The pinch point “b” is located between “a” and “c” and between the outer portion arrangement 50 and the inner portion arrangement 54 as illustrated in FIG. 11. That is, the cavity 56 as it moves in either direction along the LONG. axis from point “b” is a converging—diverging channel 58 (see FIG. 11). This converging—diverging channel 58 provides the medical professional with variable holding strength of the lip surface 16 as the cavity 56 is positioned over the patient's lips.

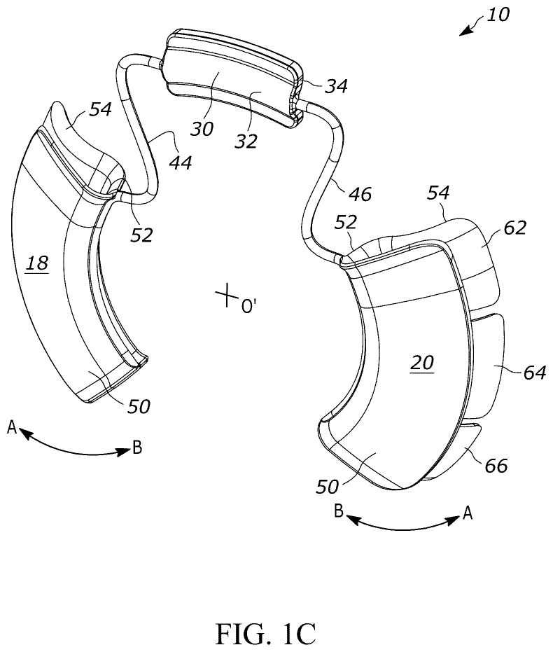

Also advantageously providing flexibility of holding strength are lines of weaknesses or slots 60 that are formed in the inner portion arrangement 54 in one example embodiment of the present disclosure. In this embodiment, the slots 60 form flexible flaps 62, 64, and 66 that allow for internal and independent movement of each flap to accommodate various wall thickness of the patient's lip surface 16 within the cavity 12 (see FIG. 3). The slots 60 forming the flaps 62, 64, and 66 on the inner portion arrangement 54 of each commissure member 18, 20 further allow for pressure release concept when a medial force is applied (with an instrument) during insertion into the cavity 12. This allows a medical device to stay in position (i.e. not fall off). Advantageously, the flaps 62, 64, and 66 alter tension distribution within the assembly 10 such that when the assembly 10 is impacted by a lateral force by instruments within the patient's mouth, the angle of the converging-diverging channel 58 is altered by movement of the outer portion arrangement 50 of first and second members 18, 20 to relieve pressure within the assembly 10, while the flaps 62, 64, and 66 remain secure within the mouth of a patient. Stated another way, the independent motion of the flaps 62, 64, and 66 allow securing to remain by flaps not contacted by instruments as one or more flaps are in contact. At least those flaps not in contact remain secured to the patient's oral cavity because the slots 60 provide independent movement.

In one example embodiment, there is a single slot 60 on each side of the assembly 10 resulting in two flaps. In another example embodiment, there are a plurality of flaps and slots greater than what is shown in the figures. The slots 60 allow the internal angle within the channel 58 to change with pressure from the medial to lateral direction when positioned within the patient's oral cavity. The number of slots 60 influences the amount of pressure, such that, the fewer number of slots increases the pressure of the hold by the channel 58. Similarly, as the number of slots increases, the pressure of the hold by the channel 58 decreases. While yet in another example embodiment, no slots are formed on the inner or outer portion, yet when the assembly is in position and a force if applied (by for example a medical device) facilitates the changing of the internal angle of the channel 58.

While in another example embodiment, the assembly 10 includes only a first member 18 and lacks any second member 20 or connecting member 30 The open cavity when installed in a patient allows for flexing between the outer and inner portions to form an increased or decreased angle about the longitudinal axis illustrated in FIG. 11 of the open cavity when a medical instrument is used such that it provides a medial or lateral force against the assembly 10.

In the illustrated example embodiment, three flaps are shown, but it should be appreciated by one of ordinary skill in the art after reviewing the present disclosure and drawings that more or less slots 60 and flaps could be used without departing from the spirit and scope of the claimed design. In one example embodiment of the present disclosure, there are two slots 60 and flaps for an assembly 10 for usage with pediatric patients. Alternatively, there may be a single flap extending from the cavity 56 which comprises a mirror image of the outer portion arrangement 50 of the first and second members 18, 20 respectively.

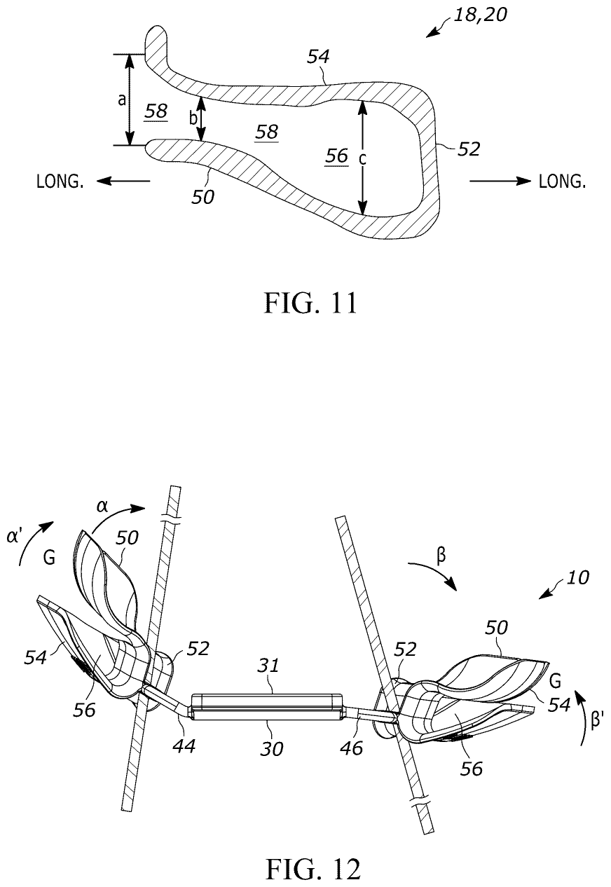

Referring now to the example embodiment in FIG. 12, two medical instruments illustrate how the channel 58 opens and closes from forces applied by a medical instrument. When a medical instrument is positioned as illustrated in FIG. 12, the opening in the channel 58 increases. That is, a force when applied to a base portion 52 of the mouth assembly 10 as shown by a medical instrument, the channel 58 reacts to open the gap G between outer and inner members 50, 54, respectively. In the example embodiment, when the force is applied by the medical instrument at the angle α the gap G opens to angle α′.

As can be seen in and appreciated from FIG. 12, the configuration, composition, and/or design of the mouth assembly 10 advantageously allows for changing of the opening of the gap G in the channel by contact from one or more medical instruments. That is, contact by a medical instrument can result in: the inner and outer portion arrangements 54, 50 respectively, moving outward to open the gap G; the inner and outer portion arrangements moving inward to close the gap G; the inner portion arrangement 54 moving inward while the outer portion arrangement 50 remains stationary; the outer portion arrangement moving inward while the inner portion arrangement remains stationary; the inner portion arrangement moving outward while the outer portion arrangement remains stationary; the outer portion arrangement moving outward while the inner portion arrangement remains stationary; the inner portion arrangement moving inward while the outer portion arrangement moves outward; the inner portion arrangement moving outward while the outer portion arrangement moves inward; and any combination thereof.

When a medical instrument is positioned as illustrated in FIG. 12, the opening in the channel 58 also advantageously decreases. That is, a force when applied to a base portion 52 of the mouth assembly 10 as shown by a medical instrument, the channel 58 reacts to close the gap G between outer and inner members 50, 54, respectively. In the example embodiment, when the force is applied by the medical instrument at the angle β the gap G closes to angle β′.

In one example embodiment, the undulating surface 31 is placed around a retractor or advantageously placed on the upper lip of the patient 14 for protection. The upper lip protection from the undulating surface 31 of the connecting member 30 can be secured with a catheter or other device. This will provide a skin/lip barrier to the upper portion of the lip of the lip surface 16. For example, a catheter is routinely used through the nostril and pulled out through the oral cavity 12 to help retract the soft palate during tonsillectomy and adenoidectomy surgeries. The undulating surface 31 of the connecting member 30 is then used as a barrier to avoid direct contact/pressure from the catheter on the skin or lip surface 16, reducing the risk of pressure injury. In one example embodiment, the trough 38 formed by the mound 36 nests the upper lip of the lip surface 16 when the assembly 10 is installed in the cavity 12, providing stabilization to the assembly 10 and protection to the patient 14.

Although the assembly 10 is described as being used in connection with a retractor or other position assist for the cavity, the assembly 10 can also be used without any such medical device. Moreover, in another alternative example embodiment, the connecting member 30 is removed and the assembly 10 comprises a single member 20 or two members 18 and 20 that are not connected and/or coupled. Yet in another example embodiment, the biasing arms 44 and 46 are linear, or arcuate, contain only a single curvature, or contain a plurality of curvatures without departing from the spirit and scope of the present disclosure. In one embodiment, the material forming the assembly 10 has a uniform thickness ranging between one quarter of one inch to one thirty-second of one inch. In another example embodiment the material forming the assembly 10 has a non-uniform thickness ranging between one quarter of one inch to one thirty-second of one inch. While in yet another example embodiment, the material thickness of the assembly 10 is approximately one sixteenth of one inch.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined by the appended claims including any amendments made during the pendency of this application and all equivalents of those claims as issued. While many of the features in the figures are selected for their function, some features have been designed for their ornamental appearance whether functional or not.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 100%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

To the extent that the materials for any of the foregoing embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A protective mouth assembly comprising:
a first member spaced from a second member, the first and second members being coupled and spaced by a connecting member;
said connecting member having a first end and a second end spaced by an undulating surface, projecting from said first and second ends are first and second biasing arms for integrally coupling said first member with said undulating surface and said second member to said undulating surface.

2. The protective mouth assembly of claim 1 wherein said first and second biasing arms comprise a radial cross section and a plurality of curvatures.

3. The protective mouth assembly of claim 1 wherein said assembly is unitarily and integrally formed from a single polymeric material.

4. The protective mouth assembly of claim 1 wherein said first and second members comprise an outer portion arrangement coupled to and spaced from an inner portion arrangement by a base portion to form a cavity therein.

5. The protective mouth assembly of claim 4 wherein said cavity further comprises a converging diverging cavity that is formed by said inner portion arrangement and said outer portion arrangement.

6. The protective mouth assembly of claim 4 wherein said inner portion further comprises a slot to form two flexible flaps contiguous to said slot.

7. A protective mouth assembly comprising:
a first member spaced from a second member, the first and second members being coupled and spaced by a connecting member;
said connecting member having a first end and a second end spaced by a connecting portion surface, projecting from said first and second ends is a biasing arm having a plurality of curvatures, the biasing arm for integrally coupling said first member with said connecting portion surface and said second member to said connecting portion surface.

8. The protective mouth assembly of claim 7 wherein said biasing arm comprises a radial cross section.

9. The protective mouth assembly of claim 7 wherein said assembly is unitarily and integrally formed from a single polymeric material.

10. The protective mouth assembly of claim 7 wherein said first and second members comprise an outer portion arrangement coupled to and space from an inner portion arrangement by a base portion to form a cavity therein.

11. The protective mouth assembly of claim 10 wherein said cavity further comprises a converging diverging cavity that is formed by said inner portion arrangement and said outer portion arrangement.

12. The protective mouth assembly of claim 10 wherein said inner portion further comprises a slot to form two flexible flaps contiguous to said slot.

13. A protective mouth assembly comprising:
a polymeric member for straddling a lip surface of a patient during use, the polymeric member comprising an outer portion arrangement coupled to and space from an inner portion arrangement by a base portion to form a cavity therein;
wherein said cavity further comprises a converging diverging cavity that is formed by said inner portion arrangement and said outer portion arrangement; and
a slot formed within the surface of said inner portion arrangement to form two flexible and independently movable flaps.

14. A protective mouth assembly comprising:
a polymeric member for straddling a lip surface of a patient during use, the polymeric member comprising an outer portion arrangement coupled to and space from an inner portion arrangement by a base portion to form a cavity therein;
a slot formed within said inner portion arrangement forming two flexible and independently movable flaps.

15. The protective mouth assembly of claim 14 wherein said slot is formed within said base portion.

16. A protective mouth assembly comprising:
a polymeric member for straddling a lip surface of a patient during use, the polymeric member comprising an outer portion arrangement coupled to and space from an inner portion arrangement by a base portion to form a cavity therein;
a continuous surface forming said inner portion arrangement such that upon engagement by a medical device during use results in a change of an inner angle of a channel formed by said cavity.

17. The protective mouth assembly of claim 16 wherein said continuous surface is discontinuous by an addition of a slot in within said inner portion forming first and second flexible flaps in the inner portion spaced by said slot.

18. The protective mouth assembly of claim 15 wherein said slot is formed within said base portion.

19. The protective mouth assembly of claim 15 wherein said cavity further comprises a converging diverging cavity that is formed by said inner portion arrangement and said outer portion arrangement.

20. The protective mouth assembly of claim 15 wherein said assembly is unitarily and integrally formed from a single polymeric material
